(19) United States
(12) Patent Application Publication
(10) Pub. No.: US 2024/0218000 A1
(43) Pub. Date: Jul. 4, 2024

(54) Diphosphites with substituents in the trans position

(71) Applicant: Evonik Oxeno GmbH & Co. KG, Marl (DE)

(72) Inventors: Anna Chiara Sale, Recklinghausen (DE); Robert Franke, Marl (DE); Dirk Fridag, Haltern am See (DE); Peter Kucmierczyk, Herne (DE); Ana Markovic, Haltern am See (DE); Armin Börner, Rostock (DE); Jens Holz, Kessin (DE)

(73) Assignee: Evonik Oxeno GmbH & Co. KG, Marl (DE)

(21) Appl. No.: 18/541,577

(22) Filed: Dec. 15, 2023

(30) Foreign Application Priority Data
Dec. 27, 2022 (EP) 22216761.1

(51) Int. Cl. C07F 9/6574; B01J 31/18; C07C 45/50

(52) U.S. Cl. CPC C07F 9/65746; B01J 31/185; C07C 45/50; B01J 2231/321; B01J 2531/822

(57) ABSTRACT

Diphosphites with substituents in the trans position and use thereof in hydroformylation.

The invention relates to diphosphites with substituents in the trans position and use thereof in hydroformylation.

Phosphorus-containing compounds play a crucial role as ligands in a multitude of reactions, e.g. in hydrogenation, in hydrocyanation and also in hydroformylation.

The reactions between olefin compounds, carbon monoxide and hydrogen in the presence of a catalyst to afford the aldehydes comprising one more carbon atom are known as hydroformylation or the oxo process. In these reactions, compounds of the transition metals of group VIII of the Periodic Table of the Elements are frequently employed as catalysts. Known ligands are, for example, compounds from the phosphine, phosphite and phosphonite classes, each containing trivalent phosphorus P^{III}. A good overview of the status of hydroformylation of olefins can be found in R. Franke, D. Selent, A. Börner, “Applied Hydroformylation”, Chem. Rev., 2012 (112), 11, 5675-5732, DOI:10.1021/cr3001803.

The technical object of the invention is to provide a compound with which an increased yield of aldehyde can be achieved in the hydroformylation of olefins.

The object is achieved by a compound according to claim 1.

Compound of formula (I):

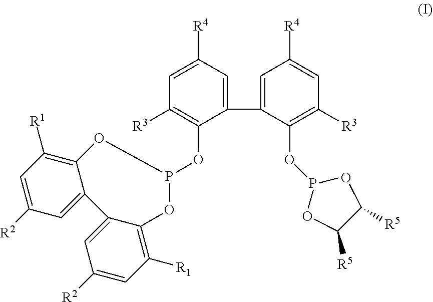

where R¹, R², R³, R⁴ are selected from: —(C₁-C₁₂)-alkyl, —O—(C₁-C₁₂)-alkyl, and
R⁵ is selected from: —(C₁-C₁₂)-alkyl, —O—(C₁-C₁₂)-alkyl, -Ph.

In one embodiment, R¹ is selected from: —CH₃, —OCH₃, —^{tert}Bu.

In one embodiment, R¹ is —^{tert}Bu.

In one embodiment, R² is selected from: —CH₃, —OCH₃, —^{tert}Bu.

In one embodiment, R² is —^{tert}Bu.

In one embodiment, R³ is selected from: —CH₃, —OCH₃, —^{tert}Bu.

In one embodiment, R³ is —^{tert}Bu.

In one embodiment, R⁴ is selected from: —CH₃, —OCH₃, —^{tert}Bu.

In one embodiment, R⁴ is —^{tert}Bu.

In one embodiment, R⁵ is selected from: —CH₃, —OCH₃, —^{tert}Bu, -Ph.

In one embodiment, R⁵ is -Ph.

In one embodiment, the compound has the structure (1):

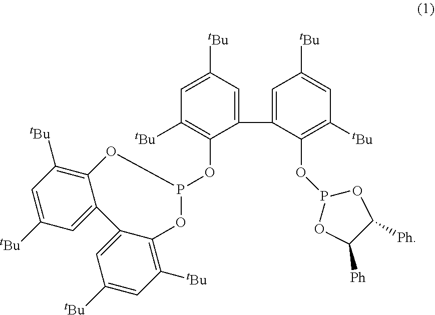 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP2O[C@H](c3ccccc3)[C@@H](c3ccccc3)O2)c(C(C)(C)C)c1]

In addition to the compound itself, a process in which the compounds described above are used is also claimed.

Process comprising the process steps of:
a) initially charging an olefin;
b) adding a compound described above;
c) adding a Rh compound;
d) feeding in H₂ and CO;
e) heating the reaction mixture from a) to d), to convert the olefin to an aldehyde.

In one variant of the process, the Rh compound is selected from: Rh(acac)(CO)₂, [(acac)Rh(COD)] (Umicore, acac=acetylacetonate anion; COD=1,5-cyclooctadiene), Rh₄CO₁₂.

In one variant of the process, the Rh compound is Rh(acac)(COD).

In one variant of the process, the H₂ and CO in process step d) are fed in at a pressure in the range from 1 to 6 MPa (10 to 60 bar).

In one variant of the process, the H₂ and CO in process step d) are fed in at a pressure in the range from 1.5 to 4.5 MPa (15 to 45 bar).

In one variant of the process, the heating of the reaction mixture in process step e) is to a temperature within a range from 80° ° C. to 160° C.

In one variant of the process, the heating of the reaction mixture in process step e) is to a temperature within a range from 100° C. to 140° C.

The invention shall be elucidated in more detail hereinbelow with reference to a working example.

### Synthesis (1): 2,4,8,10-Tetra-tert-butyl-6-((3,3′,5,5′-tetra-tert-butyl-2′-(((4R,5R)-4,5-diphenyl-1,3,2-dioxaphospholan-2-yl)oxy)-[1,1′-biphenyl]-2-yl)oxy)dibenzo[d,f][1,3,2]dioxaphosphepine

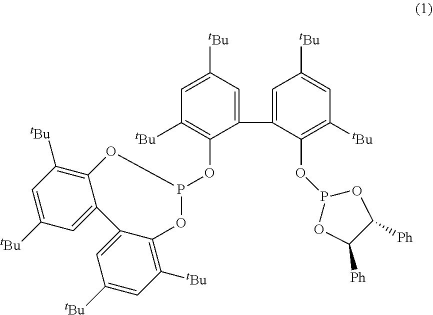 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP2O[C@H](c3ccccc3)[C@@H](c3ccccc3)O2)c(C(C)(C)C)c1]

To a solution of 3,3′,5,5′-tetra-tert-butyl-2′-((2,4,8,10-tetra-tert-butyldibenzo[d,f][1,3,2]dioxaphosphepin-6-yl)oxy)-[1,1′-biphenyl]-2-ol (0.474 g, 0.558 mmol) in THF (3 ml) were added dropwise at −20° C. a solution of n-BuLi (0.533 M solution in hexane, 1.10 ml, 0.587 mmol). After 20 min the reaction solution was allowed to warm to 0° C. and to this was then added dropwise a solution of (4R,5R)-2-chloro-4,5-diphenyl-1,3,2-dioxaphospholane (0.163 g, 0.587 mmol) in THF (3 ml). After stirring overnight at room temperature, the solvent was removed under vacuum and toluene (7 ml) added to the white residue and the undissolved constituents filtered off. The filtrate was again concentrated under vacuum and the crude product dried at 60° C. For purification, the latter was dissolved in acetonitrile at boiling point, after cooling the precipitate filtered off and the product isolated as a white solid (0.39 g, 0.357 mmol, 64% yield).

Elemental analysis (calculated for C₇₀H₉₂O₆P₂=1091.448 g/mol): C=77.22% (77.03%); H=8.57% (8.50%); P=5.50% (5.68%).

ESI-TOF HRMS: m/z=1090.6369

[M⁺+H], calculated m/z=1091.6448 (found 1091.6449)

[M⁺+Na], calculated m/z=1113.6262 (found 1113.6276)

### Synthesis (2): 2,4,8,10-Tetra-tert-butyl-6-((3,3′,5,5′-tetra-tert-butyl-2′-(((4S,5R)-4,5-diphenyl-1,3,2-dioxaphospholan-2-yl)oxy)-[1,1′-biphenyl]-2-yl)oxy)dibenzo[d,f][1,3,2]dioxaphosphepine

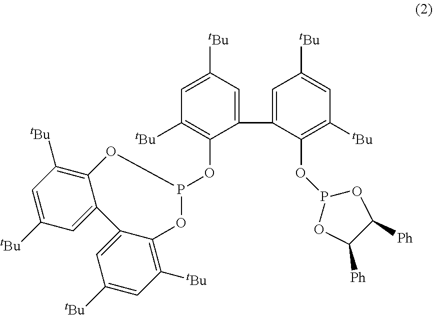 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP2O[C@H](c3ccccc3)[C@H](c3ccccc3)O2)c(C(C)(C)C)c1]

To a solution of 3,3′,5,5′-tetra-tert-butyl-2′-((2,4,8,10-tetra-tert-butyldibenzo[d,f][1,3,2]dioxaphosphepin-6-yl)oxy)-[1,1′-biphenyl]-2-ol (0.430 g, 0.506 mmol) in THF (3 ml) were added dropwise at −20° ° C. a solution of n-BuLi (0.532 M solution in hexane, 1.00 ml, 0.532 mmol). After 20 min the reaction solution was allowed to warm to 0° C. and to this was then added dropwise a solution of (4R,5S)-2-chloro-4,5-diphenyl-1,3,2-dioxaphospholane (0.148 g, 0.532 mmol) in THF (2 ml). After stirring overnight at room temperature, the solvent was removed under vacuum and toluene (6 ml) added to the white residue and the undissolved constituents filtered off. The filtrate was again concentrated under vacuum and the crude product dried at 60° C. For purification, the latter was dissolved in acetonitrile (5 ml) at boiling point, after cooling the precipitate filtered off and the product isolated as a white solid (0.22 g, 0.202 mmol, 40% yield).

Elemental analysis (calculated for C₇₀H₉₂O₆P₂=1091.448 g/mol): C=77.22% (77.03%); H=8.45% (8.50%); P=5.68% (5.68%).

ESI-TOF HRMS: m/z=1090.6369

[M⁺+H], calculated m/z=1091.6448 (found 1091.6442)

[M⁺+Na], calculated m/z=1113.6262 (found 1113.6256)

### Catalysis Experiments

The hydroformylation was conducted in a 200 ml autoclave from Premex Reactor AG, Lengau, Switzerland, equipped with pressure-retaining valve, gas flowmeter, sparging stirrer and pressure pipette. To minimize the influence of moisture and oxygen, the toluene used as solvent was purified in a Pure Solv. MD-7 system and stored under argon. The olefin cis/trans-2-pentene used as substrate (Aldrich) was heated at reflux over sodium and distilled under argon. Toluene solutions of the catalyst precursor and of the ligand were mixed in the autoclave under an argon atmosphere. Rh(acac)(COD) (Umicore, acac=acetylacetonate anion; COD=1,5-cyclooctadiene) was used as catalyst precursor. The autoclave was heated with stirring (1500 rpm) at 12 bar for a final pressure of 20 bar. After reaching the reaction temperature, the olefin was injected into the autoclave by way of a positive pressure established in the pressure pipette. The reaction was carried out at a constant pressure of 20 bar (closed-loop pressure controller from Bronkhorst, the Netherlands) over 4 h. At the end of the reaction time, the autoclave was cooled to room temperature, depressurized while stirring and purged with argon. 1 ml of reaction mixture was removed immediately after the stirrer had been switched off, diluted with 10 ml of pentane and analysed by gas chromatography: HP 5890 Series II plus, PONA, 50 m×0.2 mm×0.5 μm.

The experiment was carried out with compounds (1) and (2).

The compound (2) serves here as reference ligand.

### Results of the Catalysis Experiments

[Rh]: 100 ppm, p: 20 bar, T: 120° C.; t: 4 h; Rh:L=1:2

TABLE
Hydroformylation of cis/trans-2-pentene

| Ligand | Aldehyde yield [%] |
|---|---|
| (1)* | 100 |
| (2) | 97 |

*inventive compound

The experiments carried out demonstrate that the stated object is achieved by a compound according to the invention.

1. Compound of formula (I): 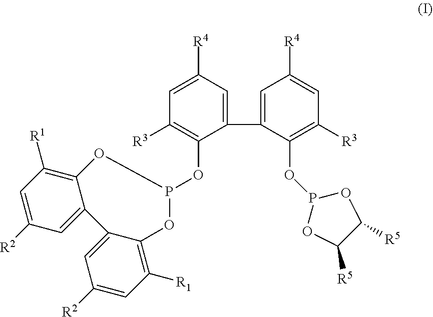
where R¹, R², R³, R⁴ are selected from: —(C₁-C₁₂)-alkyl, —O—(C₁-C₁₂)-alkyl, and
R⁵ is selected from: —(C₁-C₁₂)-alkyl, —O—(C₁-C₁₂)-alkyl, -Ph.

2. Compound according to claim 1,
wherein R¹ is selected from: —CH₃, —OCH₃, —^{tert}Bu.

3. Compound according to claim 1,
wherein R¹ is —^{tert}Bu.

4. Compound according to claim 1,
wherein R² is selected from: —CH₃, —OCH₃, —^{tert}Bu.

5. Compound according to claim 1,
wherein R² is —^{tert}Bu.

6. Compound according to claim 1,
wherein R³ is selected from: —CH₃, —OCH₃, —^{tert}Bu.

7. Compound according to claim 1,
wherein R³ is —^{tert}Bu.

8. Compound according to claim 1,
wherein R⁴ is selected from: —CH₃, —OCH₃, —^{tert}Bu.

9. Compound according to claim 1,
wherein R⁴ is —^{tert}Bu.

10. Compound according to claim 1,
wherein R⁵ is selected from: —CH₃, —OCH₃, —^{tert}Bu, -Ph.

11. Compound according to claim 1,
wherein R⁵ is -Ph.

12. Compound according to claim 1,
wherein the compound has the structure (1):

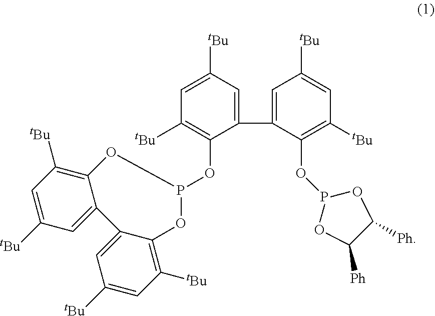 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP2O[C@H](c3ccccc3)[C@@H](c3ccccc3)O2)c(C(C)(C)C)c1]

13. Process comprising the process steps of:
a) initially charging an olefin;
b) adding a compound according to claim 1;
c) adding a Rh compound;
d) feeding in H₂ and CO;
e) heating the reaction mixture from a) to d), to convert the olefin to an aldehyde.

14. Process according to claim 13,
wherein the Rh compound is selected from: Rh(acac)(CO)₂, [(acac)Rh(COD)] (Umicore, acac=acetylacetonate anion; COD=1,5-cyclooctadiene), Rh+CO₁₂.

15. Process according to claim 13,
wherein the Rh compound is Rh(acac)(COD).
